(19) United States
(12) Patent Application Publication
(10) Pub. No.: US 2020/0054239 A1
(43) Pub. Date: Feb. 20, 2020

(54) THREE-DIMENSIONAL TOMOGRAPH

(71) Applicant: LEIBNIZ-INSTITUT FUER FESTKOERPER-UND WERKSTOFFFORSCHUNG DRESDEN E.V., Dresden (DE)

(72) Inventors: Oliver G. SCHMIDT, Dresden (DE); Mariana MEDINA SANCHEZ, Dresden (DE); Sonja Maria WEIZ, Dresden (DE)

(73) Assignee: LEIBNIZ-INSTITUT FUER FESTKOERPER-UND WERKSTOFFFORSCHUNG DRESDEN E.V., Dresden (DE)

(21) Appl. No.: 16/462,743

(22) PCT Filed: Nov. 3, 2017
(86) PCT No.: PCT/EP2017/078190

§ 371 Date: Aug. 30, 2019

(30) Foreign Application Priority Data
Nov. 22, 2016 (DE) 10 2016 223 029.8

(51) Int. Cl. A61B 5/053; A61B 5/00

(52) U.S. Cl. CPC A61B 5/0536; A61B 5/0002; A61B 2562/125; A61B 2562/028; A61B 2562/0209

(57) ABSTRACT

A three-dimensional tomograph as can be used for examining and manipulating objects on the millimeter scale or smaller, for example, for examining and manipulating biological cells, molecules or ions. The three-dimensional tomograph achieves reproducible and reliable signals in millimeter dimensions or smaller dimensions without significant signal overlays. The three-dimensional tomograph is composed of at least one three-dimensional microcomponent made of a rolled-up or folded-up layer stack of at least one carrier layer and electrodes at least located thereon for the impedance measurement. The electrodes for the impedance measurement are once or repeatedly arranged essentially on the inner or outer surface of the microcomponent in a fully surrounding manner on at least one plane, and the measurement object is located in the interior of the microcomponent and/or around the microcomponent.

The invention concerns the fields of microelectronics, materials engineering and medicine and relates to a three-dimensional tomograph as can be used for examining and manipulating objects on the millimeter scale or smaller, for example, for examining and manipulating biological cells, molecules or ions.

Various imaging methods that can determine the inner spatial structure of an object and depict it in the form of sectional images are subsumed under the term tomography. Tomographic methods can either record an individual layer or larger volumes, which can then be displayed as a series of parallel sectional images, for example. Methods which respectively record individual layers can also he used to record three-dimensional data sets, in that the object is scanned in a series of parallel cross-section images. Tomographic methods are of great importance, particularly in medical imaging (Wikipedia, German-language keyword “Tomografie”).

Electrical impedance tomography (EIT) is a relatively new, non-invasive imaging technique that is based on measurements of electrical conductivity in the human body. The basis of this technique is the observation that electrical conductivity of biological tissues is markedly different depending on the composition (absolute EIT) and/or functional condition (fitnctional or relative EIT). In addition to the basic approaches of absolute and functional EIT, in which alternating currents at a single frequency are mostly used, alternating currents of different wavelengths can also be supplied in order to address, for example, questions regarding the localization of pathological changes within a tissue type (EIT spectroscopy) (Wikipedia, German-language keyword “EIT”).

Impedance tomographs are commercially available on the macro scale, for example, for pulmonary examinations of patients.

Initial publications about miniaturized systems are also known (T. A. York et al, Meas. Sci, Technol, 2006. 17, 2119-2129; S. Dharia et al., Lab Chip 2009, 9, 3370-3377; T. Sun et al., Biosens. Bioelectron. 2010, 25, 1109-1115).

Also known is a method for measuring the distribution of electrical impedance of a multi-phase fluid, in which method an electrically conductive cylindrical annular electrode is present on the micro scale, in the interior of which a fluid is present, and the impedance is measured via contacts spatially distributed around the axis of the annular electrode on the inner and outer wall of the annular electrode (EP 1347706 A1).

According to US 20080105565 A1, a microfluid arrangement is known in which the impedance of a microfluid—with or without dispersed analytes (e.g., molecules, cells)—is measured in a microchannel by means of electrodes.

According to CA 2410743 C, a multi-channel electrode is known which comprises a plurality of electrode channels in which at least one channel has an impedance of at least 200 kΩ for receiving the electrical signals from cells and at least one channel has an impedance of less than 200 kΩ for the electrical stimulation of cells.

Roll-up technology is known for rolling up layer stacks. Layers are thereby applied on a substrate, which layers then roll up on their own during a controlled detachment from the substrate. The mechanism of the independent rolling-up is triggered, for example, by an application of the layers in a strained state and a subsequent mechanical relaxation, for example, by detaching the layers from the substrate.

The independent rolling-up of strained thin-layer capacitors when these capacitors are detached from a substrate is known according to EP 2 023 357 B1.

The formation of microtubes from stimuli-responsive materials using roll-up technology is known from publications (V. Magdanz et ale, Adv. Mater 2016, 28, 4084-4089; D. Karnaushenko et al., Adv. Mater. 2015, 27, 6797-6805). External stimuli, such as a temperature or solution composition, are thereby used to reversibly roll up a thin layer of the responsive material.

Publications regarding dielectrophoresis (DEP), which is understood as meaning the movement of a particle in a suspension due to a non-uniform electrical field, are also in existence (H. A. Pohl, J. Appl. Phys. 1951, 22, 869-871; H. A. Pohl, J. Appl. Phys. 1958, 29, 1182-1188). The use of DEP for manipulating and rotating microparticles and cells is likewise known (H. A, Pohl, J. S. Crane, Biophys, J. 1971, 11, 711-727).

A disadvantage of the solutions from the prior art is that a further miniaturization of the previously miniaturized tomographs is not possible using the known methods, since the compact contact impedance decreases as the electrode size decreases and since it overlays the signal that is to be measured. Therefore, the decrease in electrode size is only possible up to certain dimensions in order to still be able to obtain reproducible and reliable signals of the desired measured values.

The object of the present invention is to specify a three-dimensional tomograph which achieves reproducible and reliable signals in millimeter dimensions or smaller dimensions without significant signal overlays.

The object is attained by the invention disclosed in the claims. Advantageous embodiments are the subject matter of the dependent claims.

The three-dimensional tomograph according to the invention is composed of at least one three-dimensional microcomponent made of a rolled-up or folded-up layer stack of at least one carrier layer and electrodes at least located thereon for the impedance measurement, wherein the electrodes for the impedance measurement are once or repeatedly arranged essentially on the inner or outer surface of the microcomponent in a fully surrounding manner on at least one plane, and the measurement object is located in the interior of the microcomponent and/or around the microcomponent.

Advantageously, the three-dimensional microcomponent is present in a helical, tubular, elliptical, hyperbolic, toroidal, wave-like, or polygonal shape, wherein it is further advantageous if the three-dimensional microcomponent is present in the shape of a rolled-up microtube and comprises at least one winding, more advantageously up to 10 windings, of the layer stack.

Further advantageously, the electrodes for the impedance measurement are arranged at a uniform distance from one another completely across the inner perimeter of the rolled-up or folded-up microcomponent in the middle region of the microcomponent.

Likewise advantageously, the electrodes for the impedance measurement are distributed across one or more planes of the microcomponent in two or more regions, but are always arranged in a fully surrounding manner on at least one plane.

And it is also advantageous if the electrodes for the impedance measurement are arranged at a uniform distance from one another completely across the inner and/or outer perimeter in the middle region of a microcomponent.

It is also advantageous if the carrier layer is composed of one or more layers of metal, metal compounds, organic metal complexes, ceramic, semiconducting materials, biogenic materials, polymers, and/or of inorganic materials such as TiOₓ, SiOₓ or AlₓO_{y}.

It is likewise advantageous if the electrodes for the impedance measurement are composed of at least partially electrically conductive material such as copper, gold, titanium, platinum, conductive polymers, semiconductors or oxides, or of transparent electrically conductive layers such as graphene, molybdenum(IV) sulfide, indium tin oxide (ITO), poly(3,4-ethylenedioxythiophene) (PEDOT) or poly(3,4-ethylenedioxythiophene) polystyrene sulfonate (PEDOT:PSS).

It is further advantageous if the rolled-up or folded-up microcom.ponents can he rolled up or folded up and unrolled and unfolded before and/or after the impedance of an object is measured.

And it is also advantageous if additional electrodes are present for the manipulation and/or movement, such as rotation, of the measurement objects.

It is also advantageous to have sensors, actuators, signal amplifiers, or filters within the same tube, either in the internal or external layers.

And it is also advantageous if the measured signal is transmitted by means of electrically conductive contacts and connections or wirelessly.

It is likewise advantageous if the carrier and/or electrode layer is structured and/or if the roughness of the surface is modified.

With the solution according to the invention, it is for the first time possible to disclose a three-dimensional tomograph that achieves reproducible and reliable signals in millimeter dimensions or smaller dimensions without significant signal overlays.

This is achieved by a three-dimensional tomograph which is essentially composed of a three-dimensional microcomponent made of a rolled-up or folded-up layer stack, which microcomponent is provided with electrodes for the impedance measurement.

Within the scope of this invention, a three-dimensional microcomponent is to be understood as meaning a component in which at least one dimension is less than 10 mm, advantageously less than 1 mm, and further advantageously between 100 nm and 500 μm.

The three-dimensional mierocomponents can be present in a helical, tubular, elliptical, hyperbolic, toroidal, wave-like, or polygonal shape, advantageously as microtubes.

The microtube is thereby rolled up according to the invention; that is, the production of the electrodes and the contacting thereof on the at least one carrier layer has taken place in a planar state, and the microtube has subsequently rolled up independently, for example, due to a triggering of a tension or modification.

In the case of a different geometric shape, the three-dimensional microelement can also be folded up. The production of the electrodes and the contacting thereof on the at least one carrier layer thereby also take place from the planar state. In this case, the folding-up can also be initiated by a triggering of a tension or modification, for example, and the folding-up can also take place independently.

A special feature of the solution according to the invention is also that, through a reversal of the tension or modification, the microcomponent, and in particular the microtube, can once again also unroll or unfold, and that these processes of the rolling-up or folding-up and unrolling or unfolding can also be repeated multiple times in succession.

This is advantageous for the solution according to the invention, since the samples that are to be examined can, for example, also be positioned in a planar state, and can then also be rolled up or folded up and/or can be easily removed after the rolling-up or folding-up.

For the solution according to the invention, it is in principle sufficient if the microcomponent rolls up with only one complete winding or folds up into a microcomponent with the folded-up layer structure comprising the sides of the microcomponent only once. However, it is also possible that a microtube rolls up with more windings, advantageously up to 10 complete windings.

According to the invention, however, it must be kept in mind for the rolling-up or folding-up that, if at all possible, the electrodes for the impedance measurement are only arranged once in a completely surrounding manner on a plane on the inner and/or outer surface of the microcomponent.

Within the scope of the present invention, plane is to be understood as meaning a dimension in a three-dimensional space.

In the case of a microtube, the array of electrodes for the impedance measurement can be arranged between multiple windings, advantageously also at a uniform distance from one another around the perimeter of the microtube.

For other geometric shapes of the three-dimensional microcomponent, the electrodes for the impedance measurement are also advantageously arranged at a uniform distance from one another completely across the inner and/or outer perimeter of the rolled-up or folded-up microcomponent in the middle region of the microcomponent.

According to the invention, it is particularly important that electrodes for the impedance measurement are arranged around the inner and/or outer perimeter of the rolled-up or folded-up microcomponent, wherein the array of electrodes is essentially arranged only once around the inner or outer perimeter of the microcomponent. Overlaps or improper positioning of electrodes around the perimeter of the microcomponent by more than a quarter of the perimeter are not permissible.

For the three-dimensional tomograph according to the invention, it is particularly advantageous that the electrodes for the impedance measurement can be arranged such that they are distributed over one or more planes of the microcomponent in two or more regions, but always in a completely surrounding manner on at least one plane.

Over at least one dimension of the microcomponent, for example the length, which can advantageously be between 100 nm and 100 mm, one or more electrode arrays can be arranged completely on at least one plane, for example, over the height or the perimeter and/or over the width of the microcomponent. For more complex examinations, multiple electrode arrays on at least one plane, for example around the perimeter over the length of the microcomponent, are also possible and advantageous. Also, additional elements such as sensors, actuators, signal amplifiers, and/or filters can be present within the same tube, either in the internal or external layers.

With the solution according to the invention, it is also possible to perform the impedance measurement on measurement objects which are located in the microcomponent and/or also around the microcomponent.

Within the scope of this invention, measurement objects are to be understood as meaning all objects in and around the three-dimensional tomograph according to the invention that are examined at least by means of impedance measurement. If additional elements and/or electrodes are present on and/or against the carrier layer, other examinations of the measurement objects can also be conducted. In particular, additional electrodes can be present for the manipulation and/or movement, such as rotation, of the measurement objects.

The carrier layer present according to the invention can be constructed from one or more layers, wherein the carrier layer or layers are present across the full area or only partially over the perimeter of the microcomponent. Materials for the carrier layer that are, for example, made of metal, metal compounds, organic metal complexes, ceramic, semiconducting materials, biogenic materials, polymers, and/or of inorganic materials such as TiOₓ, SiOₓ or AlₓO_{y} can be present. The carrier layer can also be constructed from multiple individual layers.

If electrically conductive materials are present as a carrier layer or carrier layers, it is necessary to arrange electrically insulating intermediate layers at least in the region of the electrodes for the impedance measurement and the electrical contacts.

The electrodes for the impedance measurement and also the electrical contacts can be composed of single and/or multiple layers of at least partially electrically conductive material such as copper, gold, titanium, platinum, conductive polymers, semiconductors or oxides, or of transparent electrically conductive layers such as graphene, molyhdenum(IV) sulfide, indium tin oxide (ITO), poly(3,4-ethylenedioxythiophene) (PEDOT) or poly(3,4-ethyenedioxythiophene) polystyrene sulfonate (PEDOT:PSS), and can for example be applied by means of CVD, PVD, sputtering, electron beam evaporation, as well as spin coating, spraying, printing technologies (inkjet printing, flexography, engraving, microcontact printing, etc,), doctor blade coating, or casting.

After the production of a planar layer stack, they are rolled up or folded up into the three-dimensional component according to the invention. The rolling-up or folding-up of layer stacks can be achieved with the known roll-up technology. The layer stack thereby rolls up or folds up on its own, for example, as a result of a controlled detachment from the substrate. The mechanism of the independent rolling-up or folding-up is triggered, for example, by an application of the layers in a strained state and a subsequent mechanical relaxation, or by an application of a sacrificial layer and a subsequent at least partial removal thereof.

It is particularly advantageous for the present invention that the rolled-up or folded-up microcomponents can be rolled up or folded up and unrolled or unfolded before and/or after the impedance of an object is measured.

In this manner, for example, the measurement objects that are to be examined can be directly rolled into the three-dimensional microcomponent, or directly enclosed during the folding, and can thus be positioned in a relatively stationary manner and easily removed again from the measurement region after the measurement by rolling-up or folding-up.

With the solution according to the invention, a reproducible and reliable signal measurement can be achieved, wherein particularly as a result of the implementation of the electrodes, an accurate and non-overlaid signal measurement is enabled in the most accurate manner possible around the perimeter of the microcomponent, which measurement results in reproducible and reliable results.

However, not only is a reproducible and reliable signal measurement possible with the solution according to the invention; in addition, the measurement objects can also be stimulated and/or manipulated or the measurement objects can also be set in motion, for example, rotation. The stimulation is possible in an optical, electrical, and/or mechanical manner.

For example, the electrodes for the impedance measurement can also be used for the electrical stimulation of cells for tissue engineering. Additional electrodes, for example for dielectrophoresis, can also be integrated in and against the microcomponent so that the measurement objects can be manipulated, that is, moved, in the microcomponent. This furthermore enables the measurement of the impedance at different positions of the measurement object, so that a three-dimensional tomography can be performed.

It is likewise advantageous that the tomograph according to the invention can be produced from stimuli-responsive materials which can be precisely adapted to the object that is to be measured and, for example, also to mechanically stimulate.

Further advantageous is the small size of the three-dimensional tomograph according to the invention, which for example also renders possible the measurement of millimeter-sized or even smaller measurement objects.

In the case of the examination of cells, it is particularly advantageous that the three-dimensional tomograph according to the invention is present in a tube shape, since the in vivo environment of cells is thus simulated in an in vitro environment, and a continuous media supply and therefore a continuous flow are achieved and more realistic measurements are thus possible.

It is likewise advantageous that the measured signal can also be transmitted wirelessly in the three-dimensional tomograph according to the invention. This is particularly advantageous if the measurement object is situated in difficult-to-access locations. However, the transmission can also take place via electrically conductive contacts and connections that must also already be integrated during the production of the layer stack.

The three-dimensional array of electrodes for the impedance measurement also causes an increase in the electrode sensitivity through the near-coaxial structure that suppresses external interference. It also results in a local amplification of the electric field of the microcomponent, such that the detection limit is lowered and the measurable concentration range is increased.

As a result of the component shape, the three-dimensional tomograph according to the invention can also be integrated into microfluid systems.

The three-dimensional tomograph according to the invention can furthermore also comprise structured carrier and/or electrode layers that can carry out additional functions, and/or the roughness of the surfaces of the layers can be modified. This is particularly advantageous if the measurement object is biological cells, since certain cell types adhere better to rough surfaces and cell division is promoted. Holes in the structure can also bring about an improvement in the transport of nutrients to the cells or can function as filters.

The invention is explained below in greater detail with the aid of an exemplary embodiment.

### EXAMPLE 1

To produce a three-dimensional tomograph composed of a microtube as a microcomponent with an integrated electrode array, one sacrificial layer, one carrier layer, one electrically conductive layer, and one electrically insulating layer are applied on a quartz glass substrate with a size of 13 mm×26 mm by means of sequential lithographic and coating steps.

The sacrificial layer is composed of germanium, has a size of 150 μm×600 μm, a layer thickness of 20 nm, and is deposited at a rate of 1 Å/s in the middle of the substrate.

The carrier layer is composed of a double layer of titanium dioxide, and has a size of 250 μm×180 μm and a total thickness of 60 nm. 20 nm are thereby deposited over the sacrificial layer at a rate of 3.5 Å/s and 40 nm at a rate of 0.3 Å/s.

The electrode layer is structured to form 12 electrodes by means of lithography. The electrodes have a width of 5 μm and are arranged beneath one another in the middle at a distance of 5 μm from the shorter edge of the carrier layer. Starting from the end of the electrodes, contacts that are used for the subsequent contacting lead to the edge of the substrate. The electrode layer is composed of a 5-nm thick layer of chromium and a 10-nm thick layer of gold, which are each deposited at a rate of 1 Å/s.

The electrically insulating layer is then deposited, which layer is composed of 5 nm of silicon dioxide that is deposited at a rate of 0.3 Å/s on the electrodes and electrically conductive contacts. On each electrode, one region with a size of 5 μm×30 μm is not coated. This region is in direct contact with the measurement object during measurement.

The layer stack of the carrier layer and electrodes with the electrically insulating layer is then rolled up into a microtube. For this purpose, the sacrificial layer is dissolved in an approx. one-percent aqueous solution of hydrogen peroxide, and the microtube independently rolls up with a diameter of approx, 30 μm. The 12 electrodes are then positioned at regular intervals on the circumference on the inner jacket surface of the microtube.

The microtube is then dried at 40° C. in supercritical carbon dioxide.

The microtube is bonded onto a printed circuit board and connected in an electrically conductive manner to a potentiostat. The microtube is also connected by means of microfluid technologies and components to a reservoir in which cell medium with biological cells is present as a measurement object. The measurement objects are guided via pipelines into the interior of the microtube, where they are examined using impedance measurement.

For the measurement, an alternating current is applied to an electrode pair, and the resulting voltage is measured at the other electrodes. The supplying electrodes are varied in turn until all combinations have been measured. From the transfer impedances determined in this manner, sectional images of the interior of the microtube with the measurement objects are obtained.

1. A three-dimensional tomograph composed of at least one three-dimensional microcomponent made of a rolled-up or folded-up layer stack of at least one carrier layer and electrodes at least located thereon for the impedance measurement, wherein the electrodes for the impedance measurement are once or repeatedly arranged essentially on the inner or outer surface of the microcomponent in a fully surrounding manner on at least one plane, and the measurement object is located in the interior of the microcomponent and/or around the microcomponent.

2. The three-dimensional tomograph according to claim 1 in which the three-dimensional microcomponent is present in a helical, tubular, elliptical, hyperbolic, toroidal, wave-like, or polygonal shape.

3. The three-dimensional tomograph according to claim 2 in which the three-dimensional microcomponent is present in the shape of a rolled-up microtube and comprises at least one winding of the layer stack.

4. The three-dimensional tomograph according to claim 3 in which the rolled-up microtube is composed of one to 10 complete windings of the layer stack.

5. The three-dimensional tomograph according to claim 1 in which the electrodes for the impedance measurement are arranged at a uniform distance from one another completely across the inner perimeter of the rolled-up or folded-up microcomponent in the middle region of the microcomponent.

6. The three-diinensional tomograph according to claim 1 in which the electrodes for the impedance measurement are distributed over one or more planes of the microcomponent in two or more regions, but are always arranged in a fully surrounding manner on at least one plane.

7. The three-dimensional tomograph according to claim 1 in which the electrodes for the impedance measurement are arranged at a uniform distance from one another completely across the inner and/or outer perimeter in the middle region of a microcomponent.

8. The tree-dimensional tomograph according to claim 1 in which the carrier layer is composed of one or more layers of metal, metal compounds, organic metal complexes, ceramic, semiconducting materials, biogenic materials, polymers, and/or of inorganic materials such as TiOx, SiOx, or AlxOy.

9. The three-dimensional tomograph according to claim 1 in which the electrodes for the impedance measurement are composed of at least partially electrically conductive material such as copper, gold, titanium, platinum, conductive polymers, semiconductors or oxides, or of transparent electrically conductive layers such as graphene, molybdenum(IV) sulfide, indium tin oxide (ITO), poly(3,4-ethylenedioxythiophene) (PEDOT) or poly(3,4-ethylenedioxythiophene) polystyrene sulfonate (PEDOT:PSS).

10. The three-dimensional tomograph according to claim 1 in which the rolled-up or folded-up microcomponents can be rolled up or folded up and unrolled or unfolded before and/or after the impedance of an object is measured.

11. The three-dimensional tomograph according to claim 1 in which additional electrodes are present for the manipulation and/or movement, such as rotation, of the measurement objects.

12. The three-dimensional tomograph according to claim 1 further comprising at least one of sensors, actuators, signal amplifiers or filters being present within a same tube, either in an internal or external layer.

13. The three-dimensional tomograph according to claim 1 in which the measured signal is transmitted by means of electrically conductive contacts and connections or wirelessly.

14. The three-dimensional tomograph according to claim 1 in which the carrier and/or electrode layer is structured and/or the roughness of the surface is modified.
